(19) United States
(12) Patent Application Publication
(10) Pub. No.: US 2025/0049789 A1
(43) Pub. Date: Feb. 13, 2025

(54) METHODS OF TREATING ALOPECIA ARAETA

(71) Applicant: AbbVie Inc., North Chicago, IL (US)

(72) Inventor: Andrea Chiricozzi, Rome (IT)

(21) Appl. No.: 18/797,097

(22) Filed: Aug. 7, 2024

Related U.S. Application Data
(60) Provisional application No. 63/531,443, filed on Aug. 8, 2023.

(51) Int. Cl. A61K 31/4985; A61K 9/00; A61P 17/14

(52) U.S. Cl. CPC A61K 31/4985; A61K 9/0053; A61P 17/14

(57) ABSTRACT

The present disclosure is directed to methods for treating alopecia areata (AA) using the selective JAK1 inhibitor upadacitinib.

### CROSS-REFERENCE TO RELATED APPLICATIONS

This application claims priority to U.S. Provisional Application No. 63/531,443, filed Aug. 8, 2023, which is herein incorporated by reference in its entirety.

### FIELD OF THE DISCLOSURE

The present disclosure is directed to methods of treating alopecia areata (AA) with the selective JAK1 inhibitor, upadacitinib.

### BACKGROUND OF THE DISCLOSURE

Alopecia areata (AA) is an autoimmune disease characterized by non-scarring partial or total hair loss on the scalp or any other body area. The extent of hair loss can range from single or multiple well-defined patches to the involvement of the whole scalp (alopecia totalis) or the entire body surface.

A clinical association of alopecia areata with atopic dermatitis (AD) has been reported, with up to one-third of patients with alopecia areata who are also affected by AD. Both AD and alopecia areata are characterized by elevated levels of IgE, high circulating eosinophil count, and increased Th2 cytokine serum levels. Alopecia areata increases the risk of AD, and this risk is greatest for those with early-onset alopecia (before 10-13 years of age) and/or alopecia totalis or universalis. AD also increases the risk of severe forms of alopecia areata, with a 26-fold higher risk of developing alopecia areata in AD patients compared to healthy controls.

Despite the prevalence of alopecia areata in patients with AD, and the various approved treatments for AD, there are few systemic therapies currently available for the treatment of alopecia areata, which highlights the challenges in treating this disease, especially in severe cases. Thus, the identification of new, effective therapies for alopecia areata represents an unmet clinical need.

### SUMMARY OF THE DISCLOSURE

The present disclosure provides methods for treating alopecia areata (AA) with the selective JAK1 inhibitor, upadacitinib. The treatment methods generally comprise administering to a patient in need thereof a therapeutically effective amount of upadacitinib.

In one aspect is provided a method of treating a human patient having alopecia areata, the method comprising orally administering once daily to the patient 30 mg of upadacitinib.

In another aspect is provided a method of treating a human patient having alopecia areata, the method comprising orally administering once daily to the patient 15 mg of upadacitinib.

In some embodiments, the patient is an adult.

In another aspect is provided a method of treating alopecia areata in a human pediatric patient, the method comprising orally administering a therapeutically effective amount of upadacitinib to the pediatric patient, wherein:
if the pediatric patient has a body weight in a range from about 10 kg to less than about 20 kg:
   (i) the upadacitinib is administered twice daily at a dose of 3 mg each (3 mg BID), or
   (ii) the upadacitinib is administered twice daily at a dose of 6 mg each (6 mg BID);
if the pediatric patient has a body weight in a range from about 20 kg to less than about 30 kg:
   (i) the upadacitinib is administered twice daily at a dose of 4 mg each (4 mg BID), or
   (ii) the upadacitinib is administered twice daily at a dose of 8 mg each (8 mg BID); and
if the pediatric patient has a body weight of about 30 kg or greater:
   (i) the upadacitinib is administered twice daily at a dose of 6 mg each (6 mg BID),
   (ii) the upadacitinib is administered twice daily at a dose of 8 mg each (8 mg BID), or
   (ii) the upadacitinib is administered once daily at a dose of 15 mg (15 mg QD).

In some embodiments, the twice daily dose of upadacitinib is administered to the pediatric patient as a stable oral pharmaceutical solution.

In some embodiments, the stable oral pharmaceutical solution comprises upadacitinib, a buffer and/or pH adjusting agent, a preservative, a sweetener, and water.

In some embodiments, the oral solution comprises upadacitinib at a concentration of about 0.5 mg/mL or about 1 mg/mL.

In some embodiments, the once daily at a dose of 15 mg of upadacitinib is administered to the pediatric patient as an extended-release tablet.

In some embodiments, the method results in an AA-IGA score of 0 or 1 at 40 weeks from the first daily administration.

In some embodiments, the method results in a Severity of Alopecia Tool (SALT) score of ≤20 (less than or equal to 20% scalp hair loss) at 40 weeks from the first daily administration. In some embodiments, the method results in a SALT score of ≤10 at 40 weeks from the first daily administration. In some embodiments, the method results in a SALT score of 0 at 40 weeks from the first daily administration.

In some embodiments, the method results in SALT 50 (at least a 50% improvement [decrease] from Baseline in SALT score) at 40 weeks from the first daily administration. In some embodiments, the method results in SALT 75 (at least a 75% improvement [decrease] from Baseline in SALT score) at 40 weeks from the first daily administration. In some embodiments, the method results in a SALT 90 (at least a 90% improvement [decrease] from Baseline in SALT score) at 40 weeks from the first daily administration. In some embodiments, the method results in a SALT 100 (at least a 100% improvement [decrease] from Baseline in SALT score) at 40 weeks from the first daily administration.

In some embodiments, the method results in an AA-IGA score of 0 or 1 at 28 weeks from the first daily administration.

In some embodiments, the method results in a Severity of Alopecia Tool (SALT) score of ≤20 (less than or equal to 20% scalp hair loss) at 28 weeks from the first daily administration. In some embodiments, the method results in a SALT score of ≤10 at 28 weeks from the first daily administration. In some embodiments, the method results in a SALT score of 0 at 28 weeks from the first daily administration.

In some embodiments, the method results in SALT 50 (at least a 50% improvement [decrease] from Baseline in SALT score) at 28 weeks from the first daily administration. In some embodiments, the method results in SALT 75 (at least a 75% improvement [decrease] from Baseline in SALT score) at 28 weeks from the first daily administration. In some embodiments, the method results in a SALT 90 (at least a 90% improvement [decrease] from Baseline in SALT score) at 28 weeks from the first daily administration. In some embodiments, the method results in a SALT 100 (at least a 100% improvement [decrease] from Baseline in SALT score) at 28 weeks from the first daily administration.

In some embodiments, the method results in a Severity of Alopecia Tool (SALT) score of ≤20 (less than or equal to 20% scalp hair loss) at 24 weeks from the first daily administration. In some embodiments, the method results in a SALT score of ≤10 at 24 weeks from the first daily administration. In some embodiments, the method results in a SALT score of 0 at 24 weeks from the first daily administration.

In some embodiments, the method results in SALT 50 (at least a 50% improvement [decrease] from Baseline in SALT score) at 24 weeks from the first daily administration. In some embodiments, the method results in SALT 75 (at least a 75% improvement [decrease] from Baseline in SALT score) at 24 weeks from the first daily administration. In some embodiments, the method results in a SALT 90 (at least a 90% improvement [decrease] from Baseline in SALT score) at 24 weeks from the first daily administration. In some embodiments, the method results in a SALT 100 (at least a 100% improvement [decrease] from Baseline in SALT score) at 24 weeks from the first daily administration. In some embodiments, the method results in a Severity of Alopecia Tool (SALT) score of ≤20 (less than or equal to 20% scalp hair loss) at 12 weeks from the first daily administration. In some embodiments, the method results in a SALT score of ≤10 at 12 weeks from the first daily administration. In some embodiments, the method results in a SALT score of 0 at 12 weeks from the first daily administration.

In some embodiments, the method results in SALT 50 (at least a 50% improvement [decrease] from Baseline in SALT score) at 12 weeks from the first daily administration. In some embodiments, the method results in SALT 75 (at least a 75% improvement [decrease] from Baseline in SALT score) at 12 weeks from the first daily administration. In some embodiments, the method results in a SALT 90 (at least a 90% improvement [decrease] from Baseline in SALT score) at 12 weeks from the first daily administration. In some embodiments, the method results in a SALT 100 (at least a 100% improvement [decrease] from Baseline in SALT score) at 12 weeks from the first daily administration. In some embodiments, the method results in a Severity of Alopecia Tool (SALT) score of ≤20 (less than or equal to 20% scalp hair loss) at 8 weeks from the first daily administration. In some embodiments, the method results in a SALT score of ≤10 at 8 weeks from the first daily administration. In some embodiments, the method results in a SALT score of 0 at 8 weeks from the first daily administration.

In some embodiments, the method results in SALT 50 (at least a 50% improvement [decrease] from Baseline in SALT score) at 48 weeks from the first daily administration. In some embodiments, the method results in SALT 75 (at least a 75% improvement [decrease] from Baseline in SALT score) at 8 weeks from the first daily administration. In some embodiments, the method results in a SALT 90 (at least a 90% improvement [decrease] from Baseline in SALT score) at 8 weeks from the first daily administration. In some embodiments, the method results in a SALT 100 (at least a 100% improvement [decrease] from Baseline in SALT score) at 8 weeks from the first daily administration. In some embodiments, the method results in a Severity of Alopecia Tool (SALT) score of ≤20 (less than or equal to 20% scalp hair loss) at 4 weeks from the first daily administration. In some embodiments, the method results in a SALT score of ≤10 at 4 weeks from the first daily administration. In some embodiments, the method results in a SALT score of 0 at 4 weeks from the first daily administration.

In some embodiments, the method results in SALT 50 (at least a 50% improvement [decrease] from Baseline in SALT score) at 4 weeks from the first daily administration. In some embodiments, the method results in SALT 75 (at least a 75% improvement [decrease] from Baseline in SALT score) at 4 weeks from the first daily administration. In some embodiments, the method results in a SALT 90 (at least a 90% improvement [decrease] from Baseline in SALT score) at 4 weeks from the first daily administration. In some embodiments, the method results in a SALT 100 (at least a 100% improvement [decrease] from Baseline in SALT score) at 4 weeks from the first daily administration. In some embodiments, the method results in an achievement of ClinRO measure for Eyebrow Hair Loss of 0 or 1 at Week 24 with a ≥2 point improvement from Baseline among subjects with Baseline score ≥2.

In some embodiments, the method results in an achievement of PRO for Scalp Hair Assessment 0/1 with ≥2-point improvement (reduction) from Baseline at Week 24 among subjects with Baseline score ≥3.

In some embodiments, the method results in an achievement of PaGIC score of 1 or 2 at Week 24.

In some embodiments, the method results in an achievement of Skindex-16 AA Emotions Domain scores at Week 24.

In some embodiments, the method results in a change from Baseline in Skindex-16 AA Functioning Domain scores at Week 24.

In some embodiments, the method results in an achievement of HADS-A<8 and HADS-D<8 at Week 24 among subjects with HADS-A≥8 or HADS-D≥8 at Baseline.

In some embodiments, the method results in a change in AASIS Interference Subscale score at Week 24.

In some embodiments, the method results in a change in AASIS Symptoms Subscale score at Week 24.

In some embodiments, the method results in an achievement of ClinRO measure for Eyebrow Hair Loss of 0 or 1 at Week 12 with a ≥2 point improvement from Baseline among subjects with Baseline score ≥2.

In some embodiments, the method results in an achievement of PRO for Scalp Hair Assessment 0/1 with ≥2-point improvement (reduction) from Baseline at Week 12 among subjects with Baseline score ≥3.

In some embodiments, the method results in an achievement of PaGIC score of 1 or 2 at Week 12.

In some embodiments, the method results in an achievement of Skindex-16 AA Emotions Domain scores at Week 12.

In some embodiments, the method results in a change from Baseline in Skindex-16 AA Functioning Domain scores at Week 12.

In some embodiments, the method results in an achievement of HADS-A<8 and HADS-D<8 at Week 12 among subjects with HADS-A≥8 or HADS-D≥≥8 at Baseline.

In some embodiments, the method results in a change in AASIS Interference Subscale score at Week 12.

In some embodiments, the method results in a change in AASIS Symptoms Subscale score at Week 12.

In some embodiments, the method results in an achievement of ClinRO measure for Eyebrow Hair Loss of 0 or 1 at Week 8 with a ≥2 point improvement from Baseline among subjects with Baseline score ≥2.

In some embodiments, the method results in an achievement of PRO for Scalp Hair Assessment 0/1 with ≥2-point improvement (reduction) from Baseline at Week 8 among subjects with Baseline score ≥3.

In some embodiments, the method results in an achievement of PaGIC score of 1 or 2 at Week 8.

In some embodiments, the method results in an achievement of Skindex-16 AA Emotions Domain scores at Week 8.

In some embodiments, the method results in a change from Baseline in Skindex-16 AA Functioning Domain scores at Week 8.

In some embodiments, the method results in an achievement of HADS-A<8 and HADS-D<8 at Week 8 among subjects with HADS-A≥8 or HADS-D≥8 at Baseline.

In some embodiments, the method results in a change in AASIS Interference Subscale score at Week 8.

In some embodiments, the method results in a change in AASIS Symptoms Subscale score at Week 8.

In some embodiments, the method results in an achievement of ClinRO measure for Eyebrow Hair Loss of 0 or 1 at Week 4 with a ≥2 point improvement from Baseline among subjects with Baseline score ≥2.

In some embodiments, the method results in an achievement of PRO for Scalp Hair Assessment 0/1 with ≥2-point improvement (reduction) from Baseline at Week 4 among subjects with Baseline score ≥3.

In some embodiments, the method results in an achievement of PaGIC score of 1 or 2 at Week 4.

In some embodiments, the method results in an achievement of Skindex-16 AA Emotions Domain scores at Week 4.

In some embodiments, the method results in a change from Baseline in Skindex-16 AA Functioning Domain scores at Week 4.

In some embodiments, the method results in an achievement of HADS-A<8 and HADS-D<8 at Week 4 among subjects with HADS-A≥8 or HADS-D≥8 at Baseline.

In some embodiments, the method results in a change in AASIS Interference Subscale score at Week 4.

In some embodiments, the method results in a change in AASIS Symptoms Subscale score at Week 4.

In some embodiments, the method results in an AA-IGA score of 0 or 1 at 16 weeks from the first daily administration.

In some embodiments, the method results in a Severity of Alopecia Tool (SALT) score of 50 at 16 weeks from the first daily administration. In some embodiments, the method results in a SALT 75 at 16 weeks from the first daily administration. In some embodiments, the method results in a SALT 90 at 16 weeks from the first daily administration. In some embodiments, the method results in a SALT 100 at 16 weeks from the first daily administration.

In some embodiments, the method results in an AA-IGA score of 0 or 1 at 4 weeks from the first daily administration.

In some embodiments, the method results in a SALT 50 at 4 weeks from the first daily administration. In some embodiments, the method results in SALT 75 at 4 weeks from the first daily administration. In some embodiments, the method results in a SALT 90 at 4 weeks from the first daily administration. In some embodiments, the method results in a SALT 100 at 4 weeks from the first daily administration.

In some embodiments, the patient has moderate alopecia areata. In some embodiments, the patient has moderate or severe alopecia areata. In some embodiments, the patient has severe alopecia areata. In some embodiments, the patient has very severe alopecia areata.

In some embodiments, the alopecia areata is localized on the fronto-parietal region of the scalp and the eyebrows.

In some embodiments, the patient, prior to initiating treatment, has a serum IgE level ≥100 IU/ml.

In some embodiments, the patient has previously received treatment with a systemic agent for alopecia areata. In some embodiments, the systemic agent is cyclosporine or dupilumab. In some embodiments, the systemic agent is baricitinib.

### BRIEF DESCRIPTION OF THE DRAWINGS

FIG. 1 is a graphical depiction of therapeutic response rate over time with respect to achievement of SALT 50, SALT 75, SALT 90, and SALT 100 improvement in alopecia areata relative to baseline score for patients receiving 30 mg per day upadacitinib.

FIG. 2 is a graphical depiction of mean SALT score over time for patients exhibiting high or normal IgE serum level prior to beginning upadacitinib administration for patients receiving 30 mg per day upadacitinib.

### DETAILED DESCRIPTION OF THE DISCLOSURE

### I. Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

The term “AUC” refers to the area under the curve. AUC is the definite integral of a curve that describes the variation of a drug concentration in blood plasma as a function of time.

The term “Cₘₐₓ,” refers to the plasma concentration of the referent drug at Tₘₐₓ, expressed herein as ng/mL, produced by the oral ingestion of a single dose, or indicated number of doses, of the dosage form or pharmaceutical composition, such as the dosage forms and compositions of the present disclosure. Unless specifically indicated, Cₘₐₓ refers to the overall maximum observed concentration.

The terms “treating”, “treatment”, and “therapy” and the like, as used herein, are meant to include therapeutic measures for a disease or disorder leading to a clinically desirable or beneficial effect, including but not limited to clinically significant improvement in hair regrowth over a period of time.

As used herein, the term “pediatric patient” refers to a human patient of less than 18 years old. The terms “patient” and “subject” are used interchangeably herein.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art. Examples of pharmaceutically acceptable salts of upadacitinib may be found in WO 2017/066775, which is hereby incorporated by reference in its entirety.

### Endpoint Definitions

### Severity of Alopecia Tool (SALT) Score

The Severity of Alopecia Tool (SALT) score is the sum of the percentage hair loss in each of the four areas of the scalp (left side, right side, top and back). SALT subclasses are defined according to the S₁-S₄ categories:
S₀: no hair loss;
S₁: <25%;
S₂: 25-49%;
S₃: 50-74%;
S₄a: 75-95%;
S₄b: 96-99%;
S₅=100% of hair loss.

The SALT score is calculated by (1) multiplying the percentage of hair loss in each quadrant by the surface area of that quadrant and (2) summing the products. Scores range from 0 to 100, reflecting 0%-100% scalp hair loss. A SALT score of 1-20 indicates Mild/Limited Alopecia Areata. A SALT score of 21-49 indicates Moderate Alopecia Areata. A SALT score of 50-94 indicates Severe Alopecia Areata. A SALT score of 95-100 indicates Very Severe Alopecia Areata.

### Alopecia Areata-Investigator Global Assessment (AA-IGA)

The Alopecia Areata-Investigator Global Assessment (AA-IGA) is a measure of alopecia areata severity which includes five gradings of the extent of scalp hair loss measured using the SALT score. AA-IGA defines five levels of alopecia areata severity as:
0: 0% of scalp hair loss, “none” severity likely no hair loss;
1: 1-20%, “limited” severity requiring further evaluation towards obtaining systemic treatment benefit or potential disease control with topical and/or intralesional treatment injections;
2: 21-49%, “moderate” severity excludes the 50% of hair loss, which is found to be associated with a severe disease;
3: 50-95%; “severe”;
4: 95-100%, “very severe”, corresponding to patients with complete or almost complete hair loss, to replace the former “alopecia totalis” and “alopecia universalis” classifications.

### Eczema Area and Severity Index (EASI)

The EASI is a tool used to measure the extent (area) and severity of atopic eczema. Area score is recorded for each of the four regions of the body. The area score is the percentage of skin affected by eczema for each body region (Head and neck (Face, neck and scalp); Trunk (including genital area); Upper limbs; Lower limbs (including buttocks). The area score is based on the percentage of affected area on a scale from 1 to 6 as follows:
0: No active eczema in this region
1: 1-9%
2: 10-29%
3: 30-49%
4: 50-69%
5: 70-89%
6: 90-100%: the entire region is affected by eczema

The severity score component is the sum of the intensity scores for four signs, recorded for each of the four regions of the body noted above. The four signs are:
1. Redness (erythema, inflammation)
2. Thickness (induration, papulation, swelling-acute eczema)
3. Scratching (excoriation)
4. Lichenification (lined skin, furrowing, prurigo nodules-chronic eczema).

The average intensity of each sign in each body region is assessed as: none (0), mild (1), moderate (2) and severe (3), For each region the intensity for each of four signs is recorded and used to calculate the severity score as follows: Severity score=redness intensity+thickness intensity+scratching intensity+lichenification intensity, To calculate the EASI score, for each region, the severity score is multiplied by the area score and by, a multiplier. The multiplier is different for each body site.
Head and neck: severity score×area score×0.1 (in children 0-7 years, ×0.2)
Trunk: severity score×area score×0.3
Upper limbs: severity score×area score×0.2
Lower limbs: severity score×area score×0.4 (in children 0-7 years, ×0.3)

The total scores for each region are added to determine the final EASI score. The minimum EASI score is 0 and the maximum EASI score is 72.

### Clinician-Reported Outcome (ClinRO)

The Clinician-Reported Outcome (ClinRO) is a scale used to assess the severity of alopecia areata in eyebrows and eyelashes. ClinRO scores range from 0 (no involvement) to 3 (complete loss). A score of 0/1 indicates full coverage or minimal gaps, while a score of 2/3 indicates significant gaps or no notable hair.

### Patient-Reported Outcome (PRO)

The patient-reported outcome (PRO) alopecia score is a five-level response scale that patients use to rate their scalp hair loss. The levels are:
No missing hair: 0%
Limited: 1-20%
Moderate: 21-49%
Large: 50-94%
Nearly all or all: 95-100%

### Patient Global Impression of Change (PaGIC)

The Patient Global Impression of Change (PaGIC) scale is a self-administered questionnaire asking respondents to rate how their condition has changed since a certain point in time. The PaGIC uses a seven-point scale, ranging from very much worse to very much improved.

### Skindex-16 AA

The Skindex-16 AA is a survey consisting of 16 items reported as three domain scores: emotions (7 items), symptoms (4 items), and functioning (5 items). Scores range from 0 to 100, with higher score indicating worse QOL.

### Hospital Anxiety and Depression Scale (HADS)

The Hospital Anxiety and Depression Scale (HADS) is a self-reported rating scale of 14 items on a 4-point Likert scale (range 0-3). It is designed to measure subject anxiety (A) and depression (D). A total score of 0-7 indicates normal anxiety or depression levels, while a score of 8-10 is borderline and a score of 11-21 is abnormal.

### Alopecia Areata Symptom Impact Scale (AASIS)

The Alopecia Areata Symptom Impact Scale (AASIS) is a survey to assess the severity of alopecia areata-related symptoms and the impact of these symptoms on daily functioning. The AASIS asks patients with AA about the severity of their AA with respect to 13 items, with each item scored on a 0-10 scale (where 0 is not present and 10 is as bad as you can imagine).

### II. JAK1 Inhibition

Upadacitinib (3S,4R)-3-ethyl-4-(3H-imidazo[1,2-a]pyrrolo[2,3-e]pyrazin-8-yl)-N-(2,2,2-trifluoroethyl)pyrrolidine-1-carboxamide), or a pharmaceutically acceptable salt or solid state form thereof, is an oral Janus kinase (JAK) inhibitor that displays unique selectivity for the JAK1 receptor. Upadacitinib has the structure shown below:

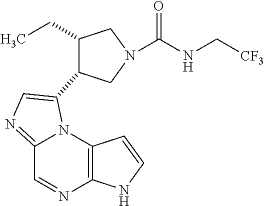

The dosage strength of upadacitinib recited in the present application is based on the weight of anhydrous freebase upadacitinib present in the active ingredient delivered to the patient. For example, a dose of “15 mg of upadacitinib” or “UPA 15 MG” refers to the 15 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “15 mg of upadacitinib” includes the administration of 15.4 mg of crystalline upadacitinib freebase hemihydrate (which includes 1/2 of a water conformer molecule per upadacitinib freebase molecule) which delivers 15 mg of anhydrous freebase upadacitinib to a patient.

### III. Treatment of Alopecia Areata (AA) with Upadacitinib

The present disclosure generally provides methods for treating a human patient having alopecia areata (AA), the methods comprising administering the selective JAK1 inhibitor upadacitinib to the patient. The disclosed methods generally comprise orally administering the upadacitinib to the patient. The upadacitinib is administered in a therapeutically effective amount. The disclosed methods generally comprise orally administering the upadacitinib to the patient daily for a period of time.

As described herein in Example 1, it has been found that upadacitinib resulted in effective and safe treatment of both AD and concomitant alopecia areata in a group of patients treated with upadacitinib 30 mg once daily. The study of Example 1 highlights the rapid and diffuse hair regrowth in AD patients treated with upadacitinib, in particular, in those with high serum IgE levels or with personal history of atopic comorbid conditions. Surprisingly, clinically meaningful hair regrowth (achievement of at least SALT 50 response) was detected in about 16% of patients after only 4 weeks of treatment in this study, despite the high severity of alopecia areata within the patient population.

### A. Adult Patient

Accordingly, in one aspect is provided a method of treating a human patient having systemic alopecia areata (AA), the method comprising orally administering once daily to the patient 30 mg of upadacitinib.

In another aspect is provided a method of treating a human patient having systemic alopecia areata (AA), the method comprising orally administering once daily to the patient 15 mg of upadacitinib.

The age of the patient may vary. In some embodiments, the patient is an adult patient (e.g., at least 18 years of age).

The method comprises orally administering upadacitinib to the patient daily for a period of time. The period of time may vary. In some embodiments, the period of time is at least 4 weeks. In some embodiments, the period of time is up to 52 weeks. In some embodiments, the period of time is at least 16 weeks, such as 16 weeks, 20 weeks, 24 weeks, 28 weeks, 36 weeks, 44 weeks, 52 weeks, 64 weeks, 76 weeks, 88 weeks, 100 weeks, or 104 weeks.

In some embodiments, the method comprises orally administering upadacitinib to the patient daily for up to 4 weeks. In some embodiments, the method comprises orally administering upadacitinib to the patient daily for up to 8 weeks. In some embodiments, the method comprises orally administering upadacitinib to the patient daily for up to 12 weeks. In some embodiments, the method comprises orally administering upadacitinib to the patient daily for up to 14 weeks. In some embodiments, the method comprises orally administering upadacitinib to the patient daily for up to 16 weeks. In some embodiments, the method comprises orally administering upadacitinib to the patient daily for up to 20 weeks. In some embodiments, the method comprises orally administering upadacitinib to the patient daily for up to 24 weeks. In some embodiments, the method comprises orally administering upadacitinib to the patient daily for up to 28 weeks. In some embodiments, the method comprises orally administering upadacitinib to the patient daily for up to 36 weeks. In some embodiments, the method comprises orally administering upadacitinib to the patient daily for up to 44 weeks. In some embodiments, the method comprises orally administering upadacitinib to the patient daily for up to 52 weeks. In some embodiments, the method comprises orally administering upadacitinib to the patient daily for up to 64 weeks. In some embodiments, the method comprises orally administering upadacitinib to the patient daily for up to 76 weeks. In some embodiments, the method comprises orally administering upadacitinib to the patient daily for up to 88 weeks. In some embodiments, the method comprises orally administering upadacitinib to the patient daily for up to 100 weeks. In some embodiments, the method comprises orally administering upadacitinib to the patient daily for up to 104 weeks.

In some embodiments, the method comprises orally administering once daily to the patient an induction dose of upadacitinib, followed by orally administering once daily to the patient a maintenance dose of upadacitinib. In some embodiments, the method comprises orally administering once daily to the patient an induction dose of 45 mg of upadacitinib, followed by orally administering once daily to the patient a maintenance dose of 15 mg of upadacitinib. In some embodiments, the method comprises orally administering once daily to the patient an induction dose of 45 mg of upadacitinib, followed by orally administering once daily to the patient a maintenance dose of 30 mg of upadacitinib. In some embodiments, the method comprises orally administering once daily to the patient an induction dose of 30 mg of upadacitinib, followed by orally administering once daily to the patient a maintenance dose of 15 mg of upadacitinib.

In some embodiments, the method results in an Alopecia Areata-Investigator Global Assessment (AA-IGA) score of 0 or 1 within 40 weeks of the first daily administration, such as within 28 weeks, 16 weeks, or 4 weeks.

In some embodiments, the method results in a Severity of Alopecia Tool (SALT) 50 within 40 weeks of the first daily administration, such as within 28 weeks, 16 weeks, or 4 weeks.

In some embodiments, the method results in a Severity of Alopecia Tool (SALT) 75 within 40 weeks of the first daily administration, such as within 28 weeks, 16 weeks, or 4 weeks.

In some embodiments, the method results in a Severity of Alopecia Tool (SALT) 90 within 40 weeks of the first daily administration, such as within 28 weeks, 16 weeks, or 4 weeks.

In some embodiments, the method results in a Severity of Alopecia Tool (SALT) 100 within 40 weeks of the first daily administration, such as within 28 weeks, 16 weeks, or 4 weeks.

The severity of the alopecia areata, prior to initiating treatment may vary. In some embodiments, the patient has moderate alopecia areata. In some embodiments, the patient has moderate or severe alopecia areata. In some embodiments, the patient has severe alopecia areata. In some embodiments, the patient has very severe alopecia areata.

The area of the patient affected by alopecia areata may vary. In some embodiments, the alopecia areata is localized on the fronto-parietal region of the scalp and the eyebrows.

In some embodiments, the patient, prior to initiating treatment, has a serum IgE level >100 IU/ml.

In some embodiments, the patient has previously received treatment with a systemic agent for alopecia areata. In some embodiments, the systemic agent is cyclosporine or dupilumab. In some embodiments, the systemic agent is baricitinib.

### B. Pediatric Patient

In some embodiments, the patient with alopecia areata is a pediatric human patient (i.e., less than about 18 years of age). In some embodiments, the dose administered to a pediatric patient may differ from that of an adult human patient.

Accordingly, in another aspect is provided a method of treating alopecia areata in a pediatric human patient. The method generally comprises administering upadacitinib to the pediatric patient as a stable oral pharmaceutical formulation or an extended-release tablet, with dosing based on body weight. The amount of upadacitinib administered, the oral dose form, and the frequency of dosing (e.g., once or twice daily) will vary based on the weight of the patient. Examples of extended-release tablets comprising upadacitinib may be found in International Patent Application Publication No. WO2017/066775, which is hereby incorporated by reference in its entirety.

In some embodiments, the pediatric patient has an age of less than 18 years. In some embodiments, the pediatric patient has an age of less than 12 years. In some embodiments, the pediatric patient has an age of less than 6 years. In some embodiments, the pediatric patient has an age in a range from about 2 to less than about 6 years, in a range from about 6 to less than about 12 years, or in a range from about 12 to less than about 18 years. In some embodiments, the pediatric patient has an age in a range from about 2 to about 18 years, such as about 2, about 3, about 4, about 5, about 6, about 7, about 8, about 9, about 10, about 11, about 12, about 13, about 14, about 15, about 16, about 17, or about 18 years of age.

In some embodiments, the pediatric patient has a body weight of at least about 10 kg. In some embodiments, the pediatric patient has a body weight from about 10 kg to less than about 30 kg, such as from about 20 to less than about 20 kg, or from about 20 to less than about 30 kg. In some embodiments, the pediatric patient has a body weight 30 kg or more.

In some embodiments, the pediatric patient has a body weight in a range from about 10 to less than about 20 kg, the method comprising administering 3 mg of upadacitinib twice daily (3 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 3 mg dose is provided BID as about 3 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL

In some embodiments, the oral solution comprises upadacitinib at a concentration of about 0.5 mg/mL and the 3 mg dose is provided BID as about 6 mL of the about 0.5 mg/mL solution.

In some embodiments, the pediatric patient has a body weight in a range from about 10 to less than about 20 kg, the method comprising administering 6 mg of upadacitinib twice daily (6 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 6 mg dose is provided BID as about 6 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL

In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight in a range from about 20 to less than about 30 kg, the method comprising administering 4 mg of upadacitinib twice daily (4 mg BID) as an oral solution. In some embodiments, the pediatric patient has a body weight in a range from about 20 to less than about 30 kg, the method comprising administering 8 mg of upadacitinib twice daily (8 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL

In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 6 mg of upadacitinib twice daily (6 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 6 mg dose is provided BID as about 6 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL

In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 15 mg of upadacitinib once daily (15 mg QD) as an extended-release tablet.

In some embodiments, the pediatric patient has a body weight of about 30 kg or greater, the method comprising administering 8 mg of upadacitinib twice daily (8 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL

In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 15 mg of upadacitinib once daily (15 mg QD) as an extended-release tablet.

The maximal concentration achieved (Cₘₐₓ) with the aforementioned dosing may vary depending on e.g., the dose, the weight of the patient, and the individual patient's metabolism of upadacitinib.

In some embodiments, when an immediate release oral solution as described herein is administered BID to the pediatric subject, a mean Cₘₐₓ for upadacitinib is achieved in a range from about 20 to about 160 ng/mL.

In some embodiments, when administered twice daily at a dose of 3 or 4 mg each (3 or 4 mg BID) to the pediatric subject, a mean Cₘₐₓ for upadacitinib is achieved in a range from about 25 to about 50 ng/mL, such as from about 25 to about 35, from about 25 to about 33, from about 25 to about 31, from about 25 to about 29, or from about 25 to about 27 ng/mL.

In some embodiments, when administered twice daily at a dose of 6 or 8 mg each (6 or 8 mg BID) to the pediatric subject, a mean Cₘₐₓ for upadacitinib is achieved in a range from about 40 to about 100 ng/mL, such as from about 40 to about 95, from about 40 to about 90, from about 40 to about 85, from about 40 to about 80, from about 40 to about 75, from about 40 to about 70, from about 40 to about 65, from about 40 to about 60, from about 40 to about 55, from about 40 to about 50, or from about 40 to about 45 ng/mL.

In some embodiments, when an extended-release 15 mg tablet as described herein is administered once daily (15 mg QD) to the pediatric subject, a mean Cₘₐₓ for upadacitinib is achieved in a range from about 45 to about 50 ng/mL, such as from about 45 to about 49, from about 45 to about 48, from about 45 to about 46, or from about 45 to about 46 ng/mL.

The mean 24-hour exposure achieved (AUC₀₋₂₄) with the aforementioned dosing may vary, depending on e.g., the dose, the weight of the patient, the fed vs. fasted condition, and the individual patient's metabolism of upadacitinib.

In some embodiments, when an immediate release oral solution as described herein is administered BID to the pediatric subject, a mean AUC₀₋₂₄ for upadacitinib is achieved in a range from about 200 to about 700 ng·h/mL.

In some embodiments, when administered twice daily at a dose of 3 or 4 mg each (3 or 4 mg BID) to the pediatric subject, a mean AUC₀₋₂₄ for upadacitinib is achieved in a range from about, such as from about 220 to about 270 ng·h/mL, such as from about 220 to about 265, from about 220 to about 260, from about 220 to about 255, from about 220 to about 250, from about 220 to about 245, from about 220 to about 240, from about 220 to about 235, from about 220 to about 230, or from about 220 to about 225 ng·h/mL.

In some embodiments, when administered twice daily at a dose of 6 or 8 mg each (6 or 8 mg BID) to the pediatric subject, a mean AUC₀₋₂₄ for upadacitinib is achieved in a range from about 340 to about 590 ng·h/mL, such as from about 340 to about 580, from about 340 to about 570, from about 340 to about 560, from about 340 to about 550, from about 340 to about 540, from about 340 to about 530, from about 340 to about 520, from about 340 to about 510, from about 340 to about 500, from about 340 to about 490, from about 340 to about 480, from about 340 to about 470, from about 340 to about 460, from about 340 to about 450, from about 340 to about 440, from about 340 to about 430, from about 340 to about 420, from about 340 to about 410, from about 340 to about 400, from about 340 to about 390, from about 340 to about 380, from about 340 to about 370, from about 340 to about 360, from about 340 to about 350, or from about 340 to about 345 ng·h/mL. In some embodiments, when administered twice daily at a dose of 6 or 8 mg each (6 or 8 mg BID) to the pediatric subject, a mean AUC₀₋₂₄ for upadacitinib is achieved in a range from about 570 to about 590 ng·h/mL, such as from about 570 to about 590, from about 570 to about 588, from about 570 to about 586, from about 570 to about 584, from about 570 to about 582, or from about 570 to about 580 ng·h/mL.

In some embodiments, when an extended-release 15 mg tablet as described herein is administered once daily (15 mg QD) to the pediatric subject, a mean AUC₀₋₂₄ for upadacitinib is achieved in a range from about 45 to about 50 ng·h/mL, such as from about 45 to about 49, about 46 to about 48, or from about 47 to about 48 ng·h/mL.

In some embodiments, the method results in an AA-IGA score of 0 or 1 at 40 weeks from the first daily administration.

In some embodiments, the method results in a Severity of Alopecia Tool (SALT) score of ≤20 (less than or equal to 20% scalp hair loss) at 40 weeks from the first daily administration. In some embodiments, the method results in a SALT score of ≤10 at 40 weeks from the first daily administration. In some embodiments, the method results in a SALT score of 0 at 40 weeks from the first daily administration.

In some embodiments, the method results in SALT 50 (at least a 50% improvement [decrease] from Baseline in SALT score) at 40 weeks from the first daily administration. In some embodiments, the method results in SALT 75 (at least a 75% improvement [decrease] from Baseline in SALT score) at 40 weeks from the first daily administration. In some embodiments, the method results in a SALT 90 (at least a 90% improvement [decrease] from Baseline in SALT score) at 40 weeks from the first daily administration. In some embodiments, the method results in a SALT 100 (at least a 100% improvement [decrease] from Baseline in SALT score) at 40 weeks from the first daily administration.

In some embodiments, the method results in an AA-IGA score of 0 or 1 at 28 weeks from the first daily administration.

In some embodiments, the method results in a Severity of Alopecia Tool (SALT) score of ≤20 (less than or equal to 20% scalp hair loss) at 28 weeks from the first daily administration. In some embodiments, the method results in a SALT score of ≤10 at 28 weeks from the first daily administration. In some embodiments, the method results in a SALT score of 0 at 28 weeks from the first daily administration.

In some embodiments, the method results in SALT 50 (at least a 50% improvement [decrease] from Baseline in SALT score) at 28 weeks from the first daily administration. In some embodiments, the method results in SALT 75 (at least a 75% improvement [decrease] from Baseline in SALT score) at 28 weeks from the first daily administration. In some embodiments, the method results in a SALT 90 (at least a 90% improvement [decrease] from Baseline in SALT score) at 28 weeks from the first daily administration. In some embodiments, the method results in a SALT 100 (at least a 100% improvement [decrease] from Baseline in SALT score) at 28 weeks from the first daily administration.

In some embodiments, the method results in a Severity of Alopecia Tool (SALT) score of ≤20 (less than or equal to 20% scalp hair loss) at 24 weeks from the first daily administration. In some embodiments, the method results in a SALT score of ≤10 at 24 weeks from the first daily administration. In some embodiments, the method results in a SALT score of 0 at 24 weeks from the first daily administration.

In some embodiments, the method results in SALT 50 (at least a 50% improvement [decrease] from Baseline in SALT score) at 24 weeks from the first daily administration. In some embodiments, the method results in SALT 75 (at least a 75% improvement [decrease] from Baseline in SALT score) at 24 weeks from the first daily administration. In some embodiments, the method results in a SALT 90 (at least a 90% improvement [decrease] from Baseline in SALT score) at 24 weeks from the first daily administration. In some embodiments, the method results in a SALT 100 (at least a 100% improvement [decrease] from Baseline in SALT score) at 24 weeks from the first daily administration. In some embodiments, the method results in a Severity of Alopecia Tool (SALT) score of ≤20 (less than or equal to 20% scalp hair loss) at 12 weeks from the first daily administration. In some embodiments, the method results in a SALT score of ≤10 at 12 weeks from the first daily administration. In some embodiments, the method results in a SALT score of 0 at 12 weeks from the first daily administration.

In some embodiments, the method results in SALT 50 (at least a 50% improvement [decrease] from Baseline in SALT score) at 12 weeks from the first daily administration. In some embodiments, the method results in SALT 75 (at least a 75% improvement [decrease] from Baseline in SALT score) at 12 weeks from the first daily administration. In some embodiments, the method results in a SALT 90 (at least a 90% improvement [decrease] from Baseline in SALT score) at 12 weeks from the first daily administration. In some embodiments, the method results in a SALT 100 (at least a 100% improvement [decrease] from Baseline in SALT score) at 12 weeks from the first daily administration. In some embodiments, the method results in a Severity of Alopecia Tool (SALT) score of ≤20 (less than or equal to 20% scalp hair loss) at 8 weeks from the first daily administration. In some embodiments, the method results in a SALT score of ≤10 at 8 weeks from the first daily administration. In some embodiments, the method results in a SALT score of 0 at 8 weeks from the first daily administration.

In some embodiments, the method results in SALT 50 (at least a 50% improvement [decrease] from Baseline in SALT score) at 48 weeks from the first daily administration. In some embodiments, the method results in SALT 75 (at least a 75% improvement [decrease] from Baseline in SALT score) at 8 weeks from the first daily administration. In some embodiments, the method results in a SALT 90 (at least a 90% improvement [decrease] from Baseline in SALT score) at 8 weeks from the first daily administration. In some embodiments, the method results in a SALT 100 (at least a 100% improvement [decrease] from Baseline in SALT score) at 8 weeks from the first daily administration. In some embodiments, the method results in a Severity of Alopecia Tool (SALT) score of ≤20 (less than or equal to 20% scalp hair loss) at 4 weeks from the first daily administration. In some embodiments, the method results in a SALT score of ≤10 at 4 weeks from the first daily administration. In some embodiments, the method results in a SALT score of 0 at 4 weeks from the first daily administration.

In some embodiments, the method results in SALT 50 (at least a 50% improvement [decrease] from Baseline in SALT score) at 4 weeks from the first daily administration. In some embodiments, the method results in SALT 75 (at least a 75% improvement [decrease] from Baseline in SALT score) at 4 weeks from the first daily administration. In some embodiments, the method results in a SALT 90 (at least a 90% improvement [decrease] from Baseline in SALT score) at 4 weeks from the first daily administration. In some embodiments, the method results in a SALT 100 (at least a 100% improvement [decrease] from Baseline in SALT score) at 4 weeks from the first daily administration. In some embodiments, the method results in an achievement of ClinRO measure for Eyebrow Hair Loss of 0 or 1 at Week 24 with a ≥2 point improvement from Baseline among subjects with Baseline score ≥2.

In some embodiments, the method results in an achievement of a PRO for Scalp Hair Assessment 0/1 with ≥2-point improvement (reduction) from Baseline at Week 24 among subjects with Baseline score ≥3.

In some embodiments, the method results in an achievement of a PaGIC score of 1 or 2 at Week 24.

In some embodiments, the method results in an achievement of a Skindex-16 AA Emotions Domain score at Week 24.

In some embodiments, the method results in a change from Baseline in a Skindex-16 AA Functioning Domain score at Week 24.

In some embodiments, the method results in an achievement of a HADS-A<8 and HADS-D<8 at Week 24 among subjects with HADS-A≥8 or HADS-D≥8 at Baseline.

In some embodiments, the method results in a change in a AASIS Interference Subscale score at Week 24.

In some embodiments, the method results in a change in a AASIS Symptoms Subscale score at Week 24.

In some embodiments, the method results in an achievement of a ClinRO measure for Eyebrow Hair Loss of 0 or 1 at Week 12 with a ≥2-point improvement from Baseline among subjects with Baseline score ≥2.

In some embodiments, the method results in an achievement of a PRO for Scalp Hair Assessment 0/1 with ≥2-point improvement (reduction) from Baseline at Week 12 among subjects with Baseline score ≥3.

In some embodiments, the method results in an achievement of PaGIC score of 1 or 2 at Week 12.

In some embodiments, the method results in an achievement of a Skindex-16 AA Emotions Domain score at Week 12.

In some embodiments, the method results in a change from Baseline in a Skindex-16 AA Functioning Domain score at Week 12.

In some embodiments, the method results in an achievement of a HADS-A<8 and HADS-D<8 at Week 12 among subjects with HADS-A≥8 or HADS-D≥8 at Baseline.

In some embodiments, the method results in a change in AASIS Interference Subscale score at Week 12.

In some embodiments, the method results in a change in AASIS Symptoms Subscale score at Week 12.

In some embodiments, the method results in an achievement of a ClinRO measure for Eyebrow Hair Loss of 0 or 1 at Week 8 with a ≥2-point improvement from Baseline among subjects with Baseline score ≥2.

In some embodiments, the method results in an achievement of a PRO for Scalp Hair Assessment 0/1 with ≥2-point improvement (reduction) from Baseline at Week 8 among subjects with Baseline score ≥3.

In some embodiments, the method results in an achievement of a PaGIC score of 1 or 2 at Week 8.

In some embodiments, the method results in an achievement of a Skindex-16 AA Emotions Domain score at Week 8.

In some embodiments, the method results in a change from Baseline in a Skindex-16 AA Functioning Domain score at Week 8.

In some embodiments, the method results in an achievement of a HADS-A<8 and a HADS-D<8 at Week 8 among subjects with HADS-A≥8 or HADS-D≥8 at Baseline.

In some embodiments, the method results in a change in AASIS Interference Subscale score at Week 8.

In some embodiments, the method results in a change in AASIS Symptoms Subscale score at Week 8.

In some embodiments, the method results in an achievement of a ClinRO measure for Eyebrow Hair Loss of 0 or 1 at Week 4 with a ≥2-point improvement from Baseline among subjects with Baseline score ≥2.

In some embodiments, the method results in an achievement of a PRO for Scalp Hair Assessment 0/1 with ≥2-point improvement (reduction) from Baseline at Week 4 among subjects with Baseline score ≥3.

In some embodiments, the method results in an achievement of a PaGIC score of 1 or 2 at Week 4.

In some embodiments, the method results in an achievement of a Skindex-16 AA Emotions Domain score at Week 4.

In some embodiments, the method results in a change from Baseline in a Skindex-16 AA Functioning Domain score at Week 4.

In some embodiments, the method results in an achievement of a HADS-A<8 and a HADS-D<8 at Week 4 among subjects with HADS-A≥8 or HADS-D≥8 at Baseline.

In some embodiments, the method results in a change in at Week 4.

In some embodiments, the method results in a change in a AASIS Symptoms Subscale score at Week 4.

In some embodiments, the method results in an AA-IGA score of 0 or 1 at 16 weeks from the first daily administration.

In some embodiments, the method results in a Severity of Alopecia Tool (SALT) 50 at 16 weeks from the first daily administration. In some embodiments, the method results in a SALT 75 at 16 weeks from the first daily administration. In some embodiments, the method results in a SALT 90 at 16 weeks from the first daily administration. In some embodiments, the method results in a SALT 100 at 16 weeks from the first daily administration.

In some embodiments, the method results in an AA-IGA score of 0 or 1 at 4 weeks from the first daily administration.

In some embodiments, the method results in a SALT 50 at 4 weeks from the first daily administration. In some embodiments, the method results in SALT 75 at 4 weeks from the first daily administration. In some embodiments, the method results in a SALT 90 at 4 weeks from the first daily administration. In some embodiments, the method results in a SALT 100 at 4 weeks from the first daily administration.

In some embodiments, the patient has moderate alopecia areata. In some embodiments, the patient has moderate or severe alopecia areata. In some embodiments, the patient has severe alopecia areata. In some embodiments, the patient has very severe alopecia areata.

In some embodiments, the alopecia areata is localized on the fronto-parietal region of the scalp and the eyebrows.

In some embodiments, the patient, prior to initiating treatment, has a serum IgE level ≥100 IU/ml.

In some embodiments, the patient has previously received treatment with a systemic agent for alopecia areata. In some embodiments, the systemic agent is cyclosporine or dupilumab. In some embodiments, the systemic agent is baricitinib.

### IV. Pharmaceutical Compositions and Routes of Administration

Upadacitinib can be administered to a human patient by itself or in pharmaceutical composition where it is mixed with biologically suitable carriers or excipient(s) at doses to treat or ameliorate a disease or condition as described herein. Mixtures of these compounds can also be administered to the patient as a simple mixture or in suitable formulated pharmaceutical compositions.

The pharmaceutical compositions of the present disclosure may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

In some embodiments, the pharmaceutical composition is a tablet dosage form. In some embodiments, tablet is a controlled-release formulation, such as an extended-release tablet dosage form (also referred to herein as a modified release or sustained release formulation). Examples of solid dosage forms comprising upadacitinib may be found in International Patent Application Publication No. WO2017/066775, which is hereby incorporated by reference in its entirety.

In some embodiments, the composition is a stable liquid pharmaceutical composition. In some embodiments, the stable liquid pharmaceutical composition is a stable oral solution. In some embodiments, the stable liquid pharmaceutical composition is a stable oral suspension. Suitable stable liquid pharmaceutical compositions comprise upadacitinib or a pharmaceutically acceptable salt or solid-state form thereof, along with excipients such as buffers, preservatives, sweeteners, flavoring agents, pH adjusting agents, solvents, and the like. In some embodiments, the stable liquid pharmaceutical composition is a stable oral pharmaceutical solution comprising upadacitinib, a buffer and/or pH adjusting agent, a preservative, a sweetener, and water. In some embodiments, the stable liquid pharmaceutical composition is a stable oral pharmaceutical suspension comprising upadacitinib, a buffer and/or pH adjusting agent, a preservative, a sweetener, and water.

The concentration of upadacitinib in the stable liquid pharmaceutical composition may vary. Due to the bitterness of upadacitinib, which is difficult to mask at higher concentrations for palatability (see, e.g., Example 5), it is generally present at about 1 mg/mL or less. In some embodiments, the stable pharmaceutical composition is an oral solution comprising: upadacitinib at a concentration in a range from about 0.3 mg/mL to about 1.2 mg/mL, such as from about 0.3 to about 0.7 mg/mL, or from about 0.8 to about 1.2 mg/mL. In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL. In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL

In particular embodiments, the oral solution has a formulation as provided in Table 1.

TABLE 1
Example formulation of upadacitinib oral solution

| Component | mg/mL |
|---|---|
| upadacitinib | 0.3-1.2 |
| citric acid, anhydrous | 0.15-0.35 |
| sodium citrate, dihydrate | 0.03-0.06 |
| sodium benzoate | 0.02-0.06 |
| sucralose | 7-11 |
| purified water | q.s to 1 mL |

### EXEMPLIFICATION

### Example 1: Efficacy and Safety of Upadacitinib in Alopecia Areata (AA)

### Study Design

AD Patients suffering from alopecia areata at the baseline visit who were treated for at least 4 weeks with upadacitinib were included in this study. According to data reported in the patient clinical report forms by each investigator, all AD patients who were concomitantly suffering from alopecia areata at the baseline visit were identified. Medical records included demographic characteristics such as age, sex, occupation, smoking habits (smoker, former smoker, or non-smoker), AD history and severity, alopecia areata history and severity, prior treatments, atopic and non-atopic comorbidities, serum IgE levels, and concomitant therapies.

Eligible patients were aged 18 to 75 years, having completed prespecified washout from prior immunomodulant systemic treatments according to criteria applied for upadacitinib trials.

Patients were treated with either 15 mg or 30 mg upadacitinib once daily (QD). The use of either 15 mg or 30 mg upadacitinib daily was based on physician's decision. All patients were initially treated with 30 mg upadacitinib daily and the dosage was maintained, except for two patients that reduced daily dose to 15 mg. No patients interrupted upadacitinib treatment throughout the observation period.

Patients were evaluated at baseline, and follow-up visits were scheduled after 4 weeks from baseline, and every 12 weeks thereafter. At baseline and at each follow-up visit, AD severity was assessed by Eczema Area and Severity Index (EASI) together with other tools. To evaluate severity and treatment response of alopecia areata, Severity of Alopecia Tool (SALT) and Alopecia Areata-Investigator Global Assessment (AA-IGA) were performed, as commonly used in daily practice. The mean SALT score was measured at the baseline, at week 4, 16, 28, 40 during upadacitinib therapy. The clinical course of alopecia areata was defined in terms of SALT 50, 75, 90, 100 (resulting in hair regrowth of 50%, 75%, 90% and 100% compared to the baseline SALT score) achieved at week 4, 16, 28, 40 during upadacitinib administration. The SALT change throughout the observation period was recorded as the difference between the baseline SALT score and the SALT score value calculated at each follow-up visit (A SALT).

### Statistical Analysis

Qualitative and quantitative variables were described as relative frequencies (%), mean and standard deviation (or medians and interquartile ranges [IR], as appropriate), respectively. The SALT 50, 75, 90, 100 achieved at week 4, 16, 28, and 40 during upadacitinib administration was analyzed according to clinical-demographic characteristics of patients using Fisher's exact test or Mann-Whitney test, as appropriate. Pearson correlation analysis was performed to assess the correlation between A SALT and i) the baseline SALT score ii) the age at the onset of alopecia iii) and/or the mean time of alopecia duration until upadacitinib administration. A multivariable regression model was built to investigate the association between A SALT and IgE levels (considered as categorical variable: high versus normal level prior to treatment initiation) and baseline SALT score (considered as continuous variable), considering gender and age at onset of alopecia as potential confounders. Statistical analyses were performed using STATA 17/BE Software (StataCorp, Texas), considering p-values<0.05 as statistically significant. Data are reported and analysed “as observed”. Thus, no missing imputation was performed.

### Results

### Demographics

Out of 118 charts of AD patients treated with upadacitinib and included in the database, 19 also suffered from concomitant alopecia areata (16.1%). Most of them were female subjects (12/19, 63.1%) with a median age of 36 years (Table 2). Concomitant autoimmune diseases were observed in 6/19 (31.6%) of patients, whereas atopic comorbidities were reported in 8/19 (42.1%) cases (Table 2). High serum IgE levels (>100 IU/ml) were detected in 6/19 (31.6%) patients. Most patients were previously treated with systemic agents including cyclosporine (12/19, 63.2%) and dupilumab (8/19, 42.1%). Mean duration of alopecia areata was 119.8±100.1 months (median [IQR]=92.7 [26.3-224.9]).

Alopecia areata was localized in all cases on the fronto-parietal region of the scalp and at the eyebrows in 18/19 (94.7%) cases. Alopecia areata universalis was observed in 14/19 (68.4%) cases (Table 2). Diagnosis of AD occurred prior to the onset of alopecia areata. Defining the mean time interval (years) between AD diagnosis and the occurrence of alopecia areata, a significantly shorter time interval for the onset of universal alopecia areata was found, compared with other clinical variants of alopecia areata (14.8±9.5 vs. 32.5±62.8 p=0.04).

According to SALT subclasses, baseline severity of alopecia areata was defined as s3 in 1/19 (5.3%), s4a in 4/19 (21.0%), s4b in 3/19 (15.8%), and s5 in 11/19 (57.9%) of cases. Based on AA-IGA scale, 4/19 (21.0%) and 11/19 (79.0%) of patients were classified as severe and very severe, respectively.

TABLE 2
Baseline demographic and clinical characteristics
of patients with AD and concomitant AA (N = 19)

| Characteristic | | N= |
|---|---|---|
| Gender, n pts (%) | Male | 7 (36.8) |
| | Female | 12 (63.2) |
| Age [median (IQR^{a})] | | 36 (26-51) |
| BMI^{b} [median (IQR^{a})] | | 23.9 (21.0-25.6) |
| Number of patients with total | >100 IU/ml, [n (%)] | 6 (31.6) |
| IgE | ≤100 IU/ml, [n (%)] | 13 (68.4) |
| Smoking, n pts (%) | Yes | 4 (21.0) |
| | No | 13 (68.4) |
| | Former smoker | 2 (10.6) |
| Alcohol consumption, | Yes | 3 (15.8) |
| n pts (%) | No | 16 (84.2) |
| AD family history, n pts (%)^{c} | Yes | 5 (41.7) |
| | No | 7 (58.3) |
| AA localization, n pts (%) | Scalp (fronto-parietal) | 19 (100) |
| | Scalp (ophiasis) | 16 (84.2) |
| | Eyebrows | 18 (94.7) |
| | Beard | 8 (42.1) |
| | Trunk and limbs | 13 (68.4) |
| | Universalis | 14 (73.7) |
| Nail involvement, n pts (%) | | 1 (5.3) |
| Mean AA duration, months ± SD^{d} | | 119.8 ± 100.1 |
| Median age of AA onset [median (IQR^{a})] | | 26[20-40] |
| Concomitant autoimmune | No | 13 (68.4) |
| disease, n pts (%) | Basedow thyroiditis | 1 (5.56) |
| | Hashimoto thyroiditis | 5 (27.8) |
| Atopic Comorbidity (asthma, | Yes | 8 (42.1) |
| rhinitis, food allergy, | No | 11 (57.9) |
| conjunctivitis), n pts (%) | | |
| ^{c}Anxiety, n pts (%) | Yes | 8 (47.0) |
| | No | 9 (52.9) |
| ^{e}Previous systemic | Cyclosporine | 12 (63.2) |
| therapies for AD, n pts (%) | Oral corticosteroids | 4 (21.0) |
| | Azathioprine | 1 (5.2) |
| | Dupilumab | 8 (42.1) |

^{a}Range interquartile
^{b}BMI: Body mass index
^{c}The sum does not match the total number of patients due to missing data
^{d}Standard deviation
^{e}The sum does not match the total due to concomitant multiple drug administration
n. pts: number of patients

### Efficacy

Positive effects of upadacitinib on alopecia areata severity were observed in terms of AA-IGA reduction, with a progressively larger percentage of patients obtaining AA-IGA 0-1 (2/19 [10.5%] at week 4, 9/17 [52.9%] at week 16, 9/14 [64.3%] at week 28, and 6/9 [66.7%] at week 40; Table 3).

A significant reduction of mean baseline SALT score (94.9±9.8) was detected as early as 4 weeks after initiation of treatment (76.4±28.5, p=0.0087) with incremental decreases that were observed over time after 16, 28, 40, and 40 weeks of treatment (Table 3). Improvement of SALT score was also measured as the difference in SALT score between each follow-up visit and baseline (A SALT), revealing a mean SALT score reduction of 18.4±26.4 points after 4 weeks, with a greater decrease at the following timepoints (Table 3).

TABLE 3
Evaluation of alopecia areata severity, clinical improvement
and treatment response during upadacitinib administration

| | | | | UPA 30 MG | | UPA 30 MG | | UPA 30 MG | | UPA 30 MG | |
|---|---|---|---|---|---|---|---|---|---|---|---|
| Items | | Baseline | | Week 4 | | Week 16 | | Week 28 | | Week 40 | |
| Number of observed | | 19 | | 19 | | 17 | | 14 | | 9 | |
| patients | | | | | | | | | | | |
| SALT score, mean ± SD^{a} | | 94.9 ± | | 76.4 ± | | 39.9 ± | | 25.8 ± | | 26.9 ± | |
| | | 9.8 | | 28.5* | | 37.7** | | 36.8*** | | 37.9**** | |
| Δ SALT, mean ± SD^{a} | | — | | −18.4 ± | | −54.3 ± | | −70.0 ± | | −70.2 ± | |
| | | | | 26.4 | | 36.8 | | 35.1 | | 36.1 | |
| AA-IGA, n | 0 | | | — | | 4/17 | (23.5) | 9/14 | (64.3) | 6/9 | (66.6) |
| pts (%) | 1 | | | 2/19 | (10.5) | 5/17 | (29.4) | — | | — | |
| | 2 | | | 3/19 | (15.8) | 2/17 | (11.8) | 1/14 | (7.1) | — | |
| | 3 | 4/19 | (21.0) | 6/19 | (31.6) | 6/17 | (35.3) | 4/14 | (28.6) | 3/9 | (33.4) |

| | | | | | | | |
|---|---|---|---|---|---|---|---|
| 4 | 15/19 | (79.0) | 8/19 | (42.1) | — | — | — |

*P value = 0.0087 for comparison between week 4 and baseline;
**P value < 0.0001 for comparison between week 16 and baseline;
***P value < 0.0001 for comparison between week 28 and baseline;
****P value < 0.0009 for comparison between week 40 and baseline.
^{a}Standard Deviation

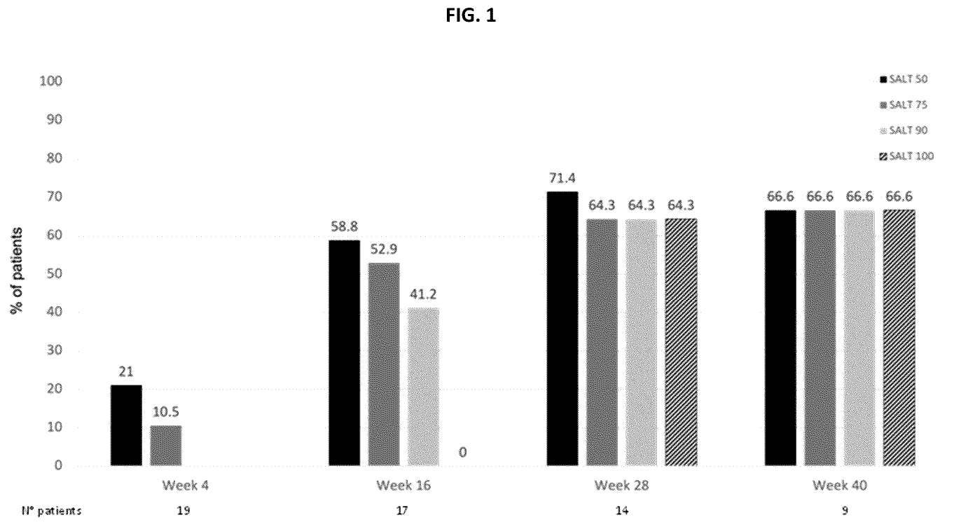

Clinical therapeutic targets (SALT 50, 75, 90, and 100 responses) were achieved by a surprising proportion of patients (3/19, 15.8%) after just 4 weeks of treatment, and this percentage markedly increased after 16 weeks of treatment. At week 16, the therapeutic response was even more striking, with more than 50% of patients achieving an AA-IGA score of 0-1 that reflected a marked reduction of SALT variation or mean SALT score. Indeed, high rates of SALT 50 and SALT 75 were achieved in over 50% of cases by week 16 (10/17, 58.8%). A graphical depiction of SALT 75 and SALT 100 responses over time is provided in FIG. 1. With reference to FIG. 1, at week 16, 52.9% (9/17) and 29.4% (5/17) of treated patients reached SALT 75 and SALT 100 responses, respectively. In patients achieving SALT 50 response during the first 16 weeks of therapy, further improvement was observed at week 28 and week 40, obtaining SALT 90 and 100 responses in most patients.

After 16 weeks of therapy there were 7/17 (41.2%) non-responder patients (<SALT 50). In these patients, no meaningful amelioration was detected thereafter.

Notably, SALT 50 and SALT 75 were more frequently observed in patients with a personal history of atopic comorbid conditions at week 16 (p=0.012 for SALT 50; and p=0.046 for SALT 75) and week 40 (p=0.028 for all clinical endpoints).

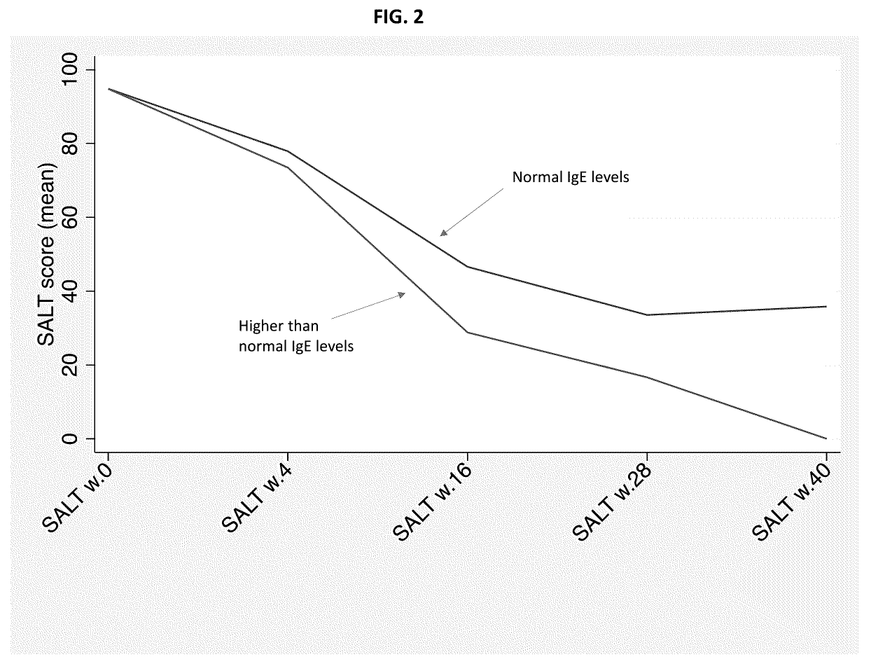

A slight positive correlation (Pearson r=0.48, p=0.0446) between changes in SALT score throughout upadacitinib treatment and the patient age at onset of alopecia areata was found and confirmed by a multivariable regression analysis, revealing a prediction of 2.1-point SALT reduction for each 1-unit increase in the age of alopecia areata onset (p=0.025), independently from baseline SALT scoring and sex (Prob>F=0.04; Adj R-squared=0.34). In addition, a significantly greater decrease of SALT score (39.0-point reduction) was found in patients with high serum levels of IgE (p=0.03). As shown in FIG. 2, compared to patients with normal serum IgE levels, those with high IgE levels had a greater improvement in SALT score (mean A SALT: −82.3±12.56 vs. −44.38±11.12, p=0.05). In contrast, neither baseline SALT score (p=0.447) nor duration of alopecia areata (p=0.378) significantly correlated with SALT score changes occurring during the observation period (Table 4).

TABLE 4
Multivariable linear regression model to understand
whether AA improvement during upadacitinib administration
could be predicted based on age at onset of alopecia,
baseline serum IgE and baseline SALT score.

| Δ SALT | Coefficient | SD^{a} | P > \|t\| | [95% CI]^{b} |
|---|---|---|---|---|
| Age at onset | 2.11 | 0.84 | 0.025 | 0.30-3.93 |
| of alopecia | | | | |
| High serum | −39.04 | 16.14 | 0.031 | −73.9-−4.16 |
| IgE levels* | | | | |
| Baseline SALT score | −0.74 | 0.91 | 0.43 | −2.70-1.22 |
| Male patients** | 20.74 | 77.65 | 0.59 | −210.18-125.32 |

*Normal serum IgE levels were considered as reference.
**Female patients were considered as reference
^{a}Standard Error
^{b}Confidence Interval

What is claimed is:

1. A method of treating a human patient having alopecia areata, the method comprising orally administering once daily to the patient 30 mg of upadacitinib.

2. A method of treating a human patient having alopecia areata, the method comprising orally administering once daily to the patient 15 mg of upadacitinib.

3. The method of claim 1 or 2, wherein the patient is an adult.

4. The method of any one of claims 1-3, wherein the method results in an AA-IGA score of 0 or 1 at 40 weeks from the first daily administration.

5. The method of any one of claims 1-4, wherein the method results in a Severity of Alopecia Tool (SALT) 50 at 40 weeks from the first daily administration.

6. The method of any one of claims 1-4, wherein the method results in SALT 75 at 40 weeks from the first daily administration.

7. The method of any one of claims 1-4, wherein the method results in a SALT 90 at 40 weeks from the first daily administration.

8. The method of any one of claims 1-4, wherein the method results in a SALT 100 at 40 weeks from the first daily administration.

9. The method of any one of claims 1-3, wherein the method results in an AA-IGA score of 0 or 1 at 28 weeks from the first daily administration.

10. The method of any one of claims 1-4, wherein the method results in a Severity of Alopecia Tool (SALT) 50 at 28 weeks from the first daily administration.

11. The method of any one of claims 1-4, wherein the method results in SALT 75 at 28 weeks from the first daily administration.

12. The method of any one of claims 1-4, wherein the method results in a SALT 90 at 28 weeks from the first daily administration.

13. The method of any one of claims 1-4, wherein the method results in a SALT 100 at 28 weeks from the first daily administration.

14. The method of any one of claims 1-3, wherein the method results in an AA-IGA score of 0 or 1 at 16 weeks from the first daily administration.

15. The method of any one of claims 1-4, wherein the method results in a Severity of Alopecia Tool (SALT) score of ≤20 at 24 weeks from the first daily administration.

16. The method of any one of claims 1-4, wherein the method results in a Severity of Alopecia Tool (SALT) 50 at 16 weeks from the first daily administration.

17. The method of any one of claims 1-4, wherein the method results in a SALT 75 at 16 weeks from the first daily administration.

18. The method of any one of claims 1-4, wherein the method results in a SALT 90 at 16 weeks from the first daily administration.

19. The method of any one of claims 1-4, wherein the method results in a SALT 100 at 16 weeks from the first daily administration.

20. The method of any one of claims 1-3, wherein the method results in an AA-IGA score of 0 or 1 at 4 weeks from the first daily administration.

21. The method of any one of claims 1-4, wherein the method results in a SALT 50 at 4 weeks from the first daily administration.

22. The method of any one of claims 1-4, wherein the method results in SALT 75 at 4 weeks from the first daily administration.

23. The method of any one of claims 1-4, wherein the method results in a SALT 90 at 4 weeks from the first daily administration.

24. The method of any one of claims 1-4, wherein the method results in a SALT 100 at 4 weeks from the first daily administration.

25. The method of any one of claims 1-24, wherein the patient has moderate or severe alopecia areata.

26. The method of any one of claims 1-24, wherein the patient has severe alopecia areata.

27. The method of any one of claims 1-26, wherein the alopecia areata is localized on the fronto-parietal region of the scalp and the eyebrows.

28. The method of any one of claims 1-27, wherein the patient, prior to initiating treatment, has a serum IgE level >100 IU/ml.

29. The method of any one of claims 1-28, wherein the patient has previously received treatment with a systemic agent for alopecia areata.

30. The method of claim 29, wherein the systemic agent is cyclosporine or dupilumab.

31. The method of claim 29, wherein the systemic agent is baricitinib.

32. A method of treating alopecia areata in a pediatric human patient, the method comprising orally administering a therapeutically effective amount of upadacitinib to the pediatric patient, wherein:
if the pediatric patient has a body weight in a range from about 10 kg to less than about 20 kg:
(i) the upadacitinib is administered twice daily at a dose of 3 mg each (3 mg BID), or
(ii) the upadacitinib is administered twice daily at a dose of 6 mg each (6 mg BID);
if the pediatric patient has a body weight in a range from about 20 kg to less than about 30 kg:
(i) the upadacitinib is administered twice daily at a dose of 4 mg each (4 mg BID), or
(ii) the upadacitinib is administered twice daily at a dose of 8 mg each (8 mg BID); and
if the pediatric patient has a body weight of about 30 kg or greater:
(i) the upadacitinib is administered twice daily at a dose of 6 mg each (6 mg BID),
(ii) the upadacitinib is administered twice daily at a dose of 8 mg each (8 mg BID), or
(ii) the upadacitinib is administered once daily at a dose of 15 mg (15 mg QD).

33. The method of claim 32, wherein the twice daily dose of upadacitinib is administered to the pediatric patient as a stable oral pharmaceutical solution.

34. The method of claim 32 or 33, wherein the stable oral pharmaceutical solution comprises upadacitinib, a buffer and/or pH adjusting agent, a preservative, a sweetener, and water.

35. The method of any one of claims 32-34, wherein the oral solution comprises upadacitinib at a concentration of about 0.5 mg/mL or about 1 mg/mL.

36. The method of any one of claims 32-35, wherein the once daily dose of upadacitinib is administered to the pediatric patient as a tablet.

37. The method of any one of claims 32-36, wherein the method results in an AA-IGA score of 0 or 1 at 40 weeks from the first daily administration.

38. The method of any one of claims 32-37, wherein the method results in a Severity of Alopecia Tool (SALT) 50 at 40 weeks from the first daily administration.

39. The method of any one of claims 32-37, wherein the method results in SALT 75 at 40 weeks from the first daily administration.

40. The method of any one of claims 32-37, wherein the method results in a SALT 90 at 40 weeks from the first daily administration.

41. The method of any one of claims 32-37, wherein the method results in a SALT 100 at 40 weeks from the first daily administration.

42. The method of any one of claims 32-36, wherein the method results in an AA-IGA score of 0 or 1 at 28 weeks from the first daily administration.

43. The method of any one of claims 32-36 or 42, wherein the method results in a Severity of Alopecia Tool (SALT) 50 at 28 weeks from the first daily administration.

44. The method of any one of claims 32-36 or 42, wherein the method results in SALT 75 at 28 weeks from the first daily administration.

45. The method of any one of claims 32-36 or 42, wherein the method results in a SALT 90 at 28 weeks from the first daily administration.

46. The method of any one of claims 32-36 or 42, wherein the method results in a SALT 100 at 28 weeks from the first daily administration.

47. The method of any one of claims 32-36, wherein the method results in an AA-IGA score of 0 or 1 at 16 weeks from the first daily administration.

48. The method of any one of claims 32-36, wherein the method results in a Severity of Alopecia Tool (SALT) score of ≤20 at 24 weeks from the first daily administration.

49. The method of any one of claims 32-36 or 47, wherein the method results in a Severity of Alopecia Tool (SALT) 50 at 16 weeks from the first daily administration.

50. The method of any one of claims 32-36 or 47, wherein the method results in a SALT 75 at 16 weeks from the first daily administration.

51. The method of any one of claims 32-36 or 47, wherein the method results in a SALT 90 at 16 weeks from the first daily administration.

52. The method of any one of claims 32-36 or 47, wherein the method results in a SALT 100 at 16 weeks from the first daily administration.

53. The method of any one of claims 32-36, wherein the method results in an AA-IGA score of 0 or 1 at 4 weeks from the first daily administration.

54. The method of any one of claims 32-36 or 53, wherein the method results in a SALT 50 at 4 weeks from the first daily administration.

55. The method of any one of claims 32-36 or 53, wherein the method results in SALT 75 at 4 weeks from the first daily administration.

56. The method of any one of claims 32-36 or 53, wherein the method results in a SALT 90 at 4 weeks from the first daily administration.

57. The method of any one of claims 32-36 or 53, wherein the method results in a SALT 100 at 4 weeks from the first daily administration.

58. The method of any one of claims 1-57, wherein the patient has moderate or severe alopecia areata.

59. The method of any one of claims 1-57, wherein the patient has severe alopecia areata.

60. The method of any one of claims 1-59, wherein the alopecia areata is localized on the fronto-parietal region of the scalp and the eyebrows.

61. The method of any one of claims 1-60, wherein the patient, prior to initiating treatment, has a serum IgE level >100 IU/ml.

62. The method of any one of claims 1-61, wherein the patient has previously received treatment with a systemic agent for alopecia areata.

63. The method of claim 62, wherein the systemic agent is cyclosporine or dupilumab.

64. The method of claim 62, wherein the systemic agent is baricitinib.
